(19) United States
(12) Patent Application Publication
(10) Pub. No.: US 2008/0095950 A1
(43) Pub. Date: Apr. 24, 2008

(54) Compositions Containing Phthalocyanine Dyes

(75) Inventors: Veronique Hall-Goulle, Dornach (CH); Athanassios Tzikas, Pratteln (CH)

Correspondence Address: Huntsman Corporation;Legal Department, 10003 Woodloch Forest Drive, The Woodlands, TX, 77380, US

(73) Assignee: Huntsman International LLC, The Woodlands, TX (US)

(21) Appl. No.: 11/661,825

(22) PCT Filed: Aug. 24, 2005
(86) PCT No.: PCT/EP05/54144

§ 371 Date: Mar. 2, 2007

(30) Foreign Application Priority Data
Sep. 3, 2004 (EP) 04104252.4
Nov. 15, 2004 (EP) 04105773.8

(51) Int. Cl. C07D 487/22; C08K 5/3417; C09B 47/04; C09B 47/18; G02B 5/20

(52) U.S. Cl. 427/532; 540/122; 8/661

(57) ABSTRACT

A composition containing (A) an alkali-soluble binder and (B) a phthalocyanine dye of formula (1), wherein Ri, R₂, R₃ and R₄ are each independently of the other Ci-Ci₂alkyl which may be unsubstituted or substituted by one or more hydroxy or mercapto groups, —X—[—O—Y]ₙ-ZH, wherein X and Y are each independently of the other C₂-C₆alkylene, Z denotes oxygen or sulphur and n is a number from 1 to 10, C₅-C₂₄aryl which may be unsubstituted or substituted by one or more hydroxy or mercapto groups, or C₆-C₃₀aralkyl which may be unsubstituted or substituted by one or more hydroxy or mercapto groups, is useful for the production of colour filters for liquid crystal displays, flat-panel displays, colour image pickup tubes, colour-copying machines etc.

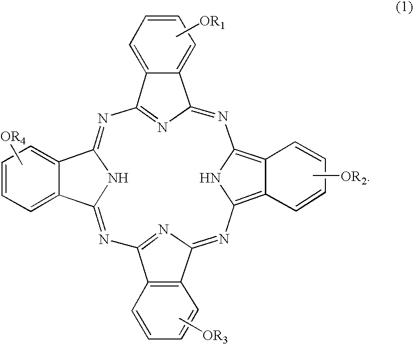

The present invention relates to compositions containing phthalocyanine dyes and their use for the production of colour filters.

Hitherto, pigments have customarily been used as colorants for colour filters employed in liquid crystal displays, flat-panel displays, colour image pickup tubes, colour-copying machines etc. However, pigment containing colour filters often suffer from inferior light transmission and low contrast because light is scattered by the pigment particles.

A further problem frequently occurs during the preparation of the pigment containing photosensitive resin. Prior to coating the resin in which the pigment is uniformly dispersed should be passed through a filter to eliminate dust and large particles whereupon clogging of the filter is sometimes caused by coagulated pigment particles.

These drawbacks can be overcome by the application of soluble dyes instead of pigments, but dyes generally do not provide sufficient thermostability and light stability. Moreover, the use of dyes in colour filters often requires a special treatment for the prevention of mixing of individual colours, for example application of protection films of polyurethane or epoxy resin or subjection of the surface of the dyed medium to chemical treatment with tannic acid or the like.

It has now been found that the problems described above can be solved by a resist formulation containing an alkali-soluble resin and a specific phthalocyanine dye.

Accordingly, the invention relates to a composition containing
(A) an alkali-soluble binder and
(B) a phthalocyanine dye of formula 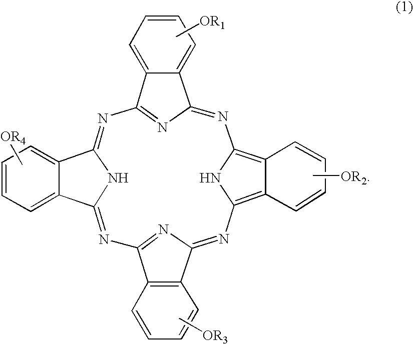
   wherein
   Ri, F?2, R3 and R₄ are each independently of the other CrCi₂alkyl which may be unsubstituted or substituted by one or more hydroxy or mercapto groups,
   —X—[—O—Y]ₙ-ZH, wherein X and Y are each independently of the other C₂-C₆alkylene, Z denotes oxygen or sulphur and n is a number from 1 to 10,
   C₅-C₂₄aryl which may be unsubstituted or substituted by one or more hydroxy or mercapto groups, or
   C₆-C₃₀aralkyl which may be unsubstituted or substituted by one or more hydroxy or mercapto groups.

The alkali-soluble binder (A) is preferably a linear organic polymer that is soluble in an organic solvent and developable with a weak alkali aqueous solution.

As the binder used in the color filter resist composition, which is soluble in an alkaline aqueous solution and insoluble in water, for example, a homopolymer of a polymerizable compound having one or more acid groups and one or more polymerizable unsaturated bonds in the molecule, or a copolymer of two or more kinds thereof, and a copolymer of one or more polymerizable compounds having one or more unsaturated bonds copolymerizable with these compounds and containing no acid group, can be used. Such compounds can be obtained by copolymerizing one or more kinds of a low molecular compound having one or more acid groups and one or more polymerizable unsaturated bonds in the molecule with one or more polymerizable compounds having one or more unsaturated bonds copolymerizable with these compounds and containing no acid group. Examples of acids groups are, a —COOH group, a —SO₃H group, a —SO₂NHCO— group, a phenolic hydroxy group, a —SO₂NH— group, and a —CO—NH—CO— group. Among those, a high molecular compound having a —COOH group is particularly preferred.

Preferably, the organic polymer binder in the color filter resist composition comprises an alkali soluble copolymer comprising, as addition polymerizable monomer units, at least an unsaturated organic acid compound such as acrylic acid, methacrylic acid and the like. It is preferred to use as a further co-monomer for the polymer binder an unsaturated organic acid ester compound such as methyl acrylate, ethyl(meth)acrylate, benzyl(meth)acrylate, styrene and the like to balance properties such as alkaline solubility, adhesion rigidity, chemical resistance etc.

The organic polymer binder can either be a random co-polymer or a block-co-polymer, for example, such as described in U.S. Pat. No. 5,368,976.

Examples of polymerizable compounds having one or more acid group and one or more polymerizable unsaturated bond in the molecule include the following compounds:

Examples of the polymerizable compounds having one or more —COOH groups and one or more polymerizable unsaturated bonds in a molecule are (meth)acrγlic acid, 2-carboxyethyl(meth)acrylic acid, 2-carboxypropyl(meth)acrylic acid, crotonic acid, cinnamic acid, mono[2-(meth)acryloyloxyethyl]succinate, mono[2-(meth)acryloyloxyethyl]adipate, mono[2-(meth)-acryloyloxyethyl]phthalate, mono[2-(meth)acryloyloxyethyl]hexahydrophthalate, mono[2-(meth)acryloyloxyethyl]maleate, mono[2-(meth)acryloyloxypropyl]succinate, mono[2-(meth)acryloyloxypropyl]adipate, mono[2-(meth)acryloyloxypropyl]phthalate, mono[2-(meth)acryloyloxypropyl]hexahydrophthalate, mono[2-(meth)acryloyloxypropyl]maleate, mono[2-(meth)acryloyloxybutyl]succinate, mono[2-(meth)acryloyloxybutyl]adipate, mono[2-(meth)acryloyloxybutyl]phthalate, mono[2-(meth)acryloyloxybutyl]hexahydrophthalate, mono[2-(meth)acryloyloxybutyl]maleate, 3-(alkylcarbamoyl)acrylic acid, α-chloroacrylic acid, maleic acid, monoesterified maleic acid, fumaric acid, itaconic acid, citraconic acid, mesaconic acid, maleic anhydride, and ω-carboxypolycaprolactone mono(meth)acrylate.

Vinylbenzenesulfonic acid and 2-(meth)acrylamide-2-methylpropanesulfonic acid are examples of the polymerizable compounds having one or more —SO₃H groups and one or more polymerizable unsaturated bonds.

N-methylsulfonyl(meth)acrylamide, N-ethylsulfonyl(meth)acrylamide, N-phenylsulfonyl(meth)acrylamide, and N-(p-methylphenylsulfonyl)(meth)acrylamide are examples of the polymerizable compounds having one or more —SO₂NHCO— groups and one or more polymerizable unsaturated bonds.

Examples of polymerizable compounds having one or more phenolic hydroxy groups and one or more polymerizable unsaturated bonds in a molecule include hydroxyphenyl(meth)acrylamide, dihydroxyphenyl(meth)acrylamide, hydroxyphenyl-carbonyloxyethyl(meth)acrylate, hydroxyphenyloxyethyl(meth)acrylate, hydroxyphenylthioethyl(meth)acrylate, dihydroxyphenylcarbonyloxyethyl(meth)acrylate, dihydroxyphenyloxyethyl(meth)acrylate, and dihydroxyphenylthioethyl(meth)acrylate.

Examples of the polymerizable compound having one or more —SO₂NH— groups and one or more polymerizable unsaturated bonds in the molecule include compounds represented by formula (a) or (b):
CH₂═CHA₁-Y₁-A₂-SO₂—NH-A₃ (a)
CH₂═CHA₄-Y₂-A₅-NH—SO₂-A₆ (b)
wherein Y₁ and Y₂ each represents —COO—, —CONA₇-, or a single bond; A₁ and A₄ each represents H or CH₃; A₂ and A₅ each represents CᵣC₁₂alkylene optionally having a substituent, cycloalkylene, arylene, or aralkylene, or C₂-C₁₂alkylene into which an ether group and a thioether group are inserted, cycloalkylene, arylene, or aralkylene; A₃ and A₆ each represents H, CrC₁₂alkyl optionally having a substituent, a cycloalkyl group, an aryl group, or an aralkyl group; and A₇ represents H, CᵣC₁₂alkyl optionally having a substituent, a cycloalkyl group, an aryl group, or an aralkyl group.

The polymerizable compounds having one or more —CO—NH—CO— group and one or more polymerizable unsaturated bond include maleimide and N-acryloyl-acrylamide. These polymerizable compounds become the high molecular compounds comprising a —CO—NH—CO— group, in which a ring is formed together with a primary chain by polymerization. Further, a methacrylic acid derivative and an acrylic acid derivative each having a —CO—NH—CO— group can be used as well. Such methacrylic acid derivatives and the acrylic acid derivatives include, for example, a methacrylamide derivative such as N-acetylmethacrylamide, N-propionylmethacrylamide, N-butanoylmethacrylamide, N-pentanoylmethacrylamide, N-decanoylmethacrylamide, N-dodecanoylmethacrylamide, N-benzoylmethacrylamide, N-(p-methylbenzoyl)methacryl-amide, N-(p-chlorobenzoyl)methacrylamide, N-(naphthyl-carbonyl)methacrylamide, N-(phenylacetyl)methacryl-amide, and 4-methacryloylaminophthalimide, and an acrylamide derivative having the same substituent as these. These polymerizable compounds polymerize to be compounds having a —CO—NH—CO— group in a side chain.

Examples of polymerizable compounds having one or more polymerizable unsaturated bond and containing no acid group include a compound having a polymerizable unsaturated bond, selected from esters of (meth)acrylic acid, such as methyl(meth)acrylate, ethyl(meth)acrylate, propyl(meth)acrylate, butyl(meth)acrylate, tetrahydrofurfuryl(meth)acrylate, benzyl(meth)acrylate, 2-ethylhexyl(meth)acrylate, hydroxyethyl(meth)acrylate, hydroxypropyl(meth)acrylate, hydroxybutyl(meth)acrylate, glycerol mono(meth)acrylate, dihydroxypropyl(meth)acrylate, allyl(meth)acrylate, cyclohexyl(meth)acrylate, phenyl(meth)acrylate, methoxyphenyl(meth)acrylate, methoxyethyl(meth)acrylate, phenoxyethyl(meth)acrylate, methoxydiethyleneglycol(meth)acrylate, methoxytriethyleneglycol(meth)acrylate, methoxypropyl(meth)acrylate, methoxydipropyleneglycol(meth)acrylate, isobornyl meth(acrylate), dicyclopentadienyl(meth)acrylate, 2-hydroxy-3-phenoxypropyl(meth)acrylate, tricyclo[5.2.1.0^{2.}^{⁶}]decan-8-yl(meth)acrylate, aminoethyl(meth)acrylate, N,N-dimethylaminoethyl(meth)acrylate, aminopropyl(meth)acrylate, N,N-dimethylaminopropyl(meth)acrylate, glycidyl(meth)acrylate, 2-methylglycidyl(meth)acrylate, 3,4-epoxybutyl(meth)acrylate, 6,7-epoxyheptyl(meth)acrylate; vinyl aromatic compounds, such as styrene, α-methylstyrene, vinyltoluene, p-chlorostyrene, polychlorostyrene, fluorostyrene, bromostyrene, ethoxymethyl styrene, methoxystyrene, 4-methoxy-3-methystyrene, dimethoxystyrene, vinylbenzyl methyl ether, vinylbenzyl glycidyl ether, indene, 1-methylindene; vinyl or allyl esters, such as vinyl acetate, vinyl propionate, vinyl butylate, vinyl pivalate, vinyl benzoate, vinyl trimethylacetate, vinyl diethylacetate, vinyl barate, vinyl caproate, vinyl chloroacetate, vinyl dichloroacetate, vinyl methoxyacetate, vinyl butoxyacetate, vinyl phenylacetate, vinyl acetate, vinyl acetoacetate, vinyl lactate, vinyl phenylbutylate, vinyl cyclohexylcarboxylate, vinyl salicylate, vinyl chlorobenzoate, vinyl tetrachlorobenzoate, vinyl naphthoate, allyl acetate, allyl propionate, allyl butylate, allyl pivalate, allyl benzoate, allyl caproate, allyl stearate, allyl acetoacetate, allyl lactate; vinyl or allyl ethers, such as vinyl methyl ether, vinyl ethyl ether, vinyl hexyl ether, vinyl octyl ether, vinyl ethylhexyl ether, vinyl methoxyethyl ether, vinyl ethoxyethyl ether, vinyl chloroethyl ether, vinyl hydroxyethyl ether, vinyl ethylbutyl ether, vinyl hydroxyethoxyethyl ether, vinyl dimethylaminoethyl ether, vinyl diethylaminoethyl ether, vinyl butylaminoethyl ether, vinyl benzyl ether, vinyl tetrahydrofurfuryl ether, vinyl phenyl ether, vinyl tolyl ether, vinyl chlorophenyl ether, vinyl chloroethyl ether, vinyl dichlorophenyl ether, vinyl naphthyl ether, vinyl anthryl ether, allyl glycidyl ether; amide type unsaturated compounds, such as (meth)acrylamide, N,N-dimethyl(meth)acrylamide, N,N-diethyl(meth)acrylamide, N,N-dibutyl(meth)acrylamide, N,N-diethylhexyl(meth)acrylamide, N,N-dicyclohexyl(meth)acrylamide, N,N-diphenyl(meth)acrylamide, N-methyl-N-phenyl(meth)acrylamide, N-hydroxyethyl-N-methyl(meth)acrylamide, N-methyl(meth)acrylamide, N-ethyl(meth)acrylamide, N-propyl(meth)acrylamide, N-butyl(meth)acrylamide, N-hydroxyethyl(meth)acrylamide, N-heptyl(meth)acrylamide, N-octyl(meth)acrylamide, N-ethyhexyl(meth)acrylamide, N-hydroxyethyl(meth)acrylamidecyclohexyl, N-benzyl(meth)acrylamide, N-phenyl(meth)acrylamide, N-tolyl(meth)acrylamide, N-hydroxyphenyl(meth)acrylamide, N-naphthyl(meth)acrylamide, N-phenylsulfonyl(meth)acrylamide, N-methylphenylsulfonyl(meth)acrylamide and N-(meth)acryloylmorpholine _{J}diacetone acrylamide, N-methylol acrylamide, N-butoxyacrylamide; polyolefin type compounds, such as butadiene, isoprene, chloroprene and the like; (meth)acrylonitrile, methyl isopropenyl ketone, maleimide, N-phenylmaleimide, N-methylphenylmaleimide, N-methoxyphenylmaleimide, N-cyclohexylmaleimide, N-alkylmaleimide, maleic anhydride, polystyrene macromonomer, polymethyl(meth)acrylate macromonomer, polybutyl(meth)acrylate macromonomer; crotonates, such as butyl crotonate, hexyl crotonate, glycerine monocrotonate; and itaconates, such as dimethyl itaconate, diethyl itaconate, dibutyl itaconate; and maleates or fumarates, such as dimethyl mareate, dibutyl fumarate.

Preferable examples of copolymers are copolymers of methyl(meth)acrylate and (meth)acrylic acid, copolymers of benzyl(meth)acrylate and (meth)acrylic acid, copolymers of methyl(meth)acrylate/, ethyl(meth)acrylate and (meth)acrylic acid, copolymers of benzyl(meth)acrylate, (meth)acrylic acid and styrene, copolymers of benzyl(meth)acrylate, (meth)acrylic acid and 2-hydroxyethyl(meth)acrylate, copolymers of methyl(meth)acrylate/, butyl(meth)acrylate, (meth)acrylic acid and styrene, copolymers of methyl(meth)acrylate, benzyl(meth)acrylate, (metha)crylic acid and hydroxyphenyl(meth)acrylate, copolymers of methyl(meth)acrylate, (metha)crylic acid and polymethyl(meth)acrylate macromonomer, copolymers of benzyl(meth)crylate, (metha)crylic acid and polymethyl(meth)acrylate macromonomer, copolymers of tetrahydrofurfuryl(meth)acrylate, styrene and (meth)acrylic acid, copolymers of methyl(meth)acrylate, (meth)acrylic acid and polystyrene macromonomer, copolymers of benzyl(meth)acrylate, (meth)acrylic acid and polystyrene macromonomer, copolymers of benzyl(meth)acrylate, (meth)acrylic acid, 2-hydroxyethyl(meth)acrylate and polystyrene macromonomer, copolymers of benzyl(meth)acrylate, (meth)acrylic acid, 2-hydroxypropyl(meth)acrylate and polystyrene macromonomer, copolymers of benzyl(meth)acrylate, (meth)acrylic acid, 2-hydroxy-3-phenoxypropyl(meth)acrylate and polymethyl(meth)acrylate macromonomer, copolymers of methyl(meth)acrylate, (meth)acrylic acid, 2-hydroxyethyl(meth)acrylate and polystyrene macromonomer, copolymers of benzyl(meth)acrylate, (metha)crylic acid, 2-hydroxyethyl(meth)acrylate and polymethyl(meth)acrylate macromonomer, copolymers of N-phenylmaleimide, benzyl(meth)acrylate, (metha)crylic acid and styrene, copolymers of benzyl(meth)acrylate, (meth)acrylic acid, N-phenylmaleimide, mono-[2-(meth)acryloyloxyethyl]succinate and styrene, copolymers of allyl(meth)acrylate, (meth)acrylic acid, N-phenylmaleimide, mono-[2-(meth)acryloyloxyethyl]succinate and styrene, copolymers of benzyl(meth)acrylate, (meth)acrylic acid, N-phenylmaleimide, glycerol mono(meth)acrylate and styrene, copolymers of benzyl(meth)acrylate, ω-carboxypolycaprolactone mono(meth)acrylate, (meth)acrylic acid, N-phenylmaleimide, glycerol mono(meth)acrylate and styrene, and copolymers of benzyl(meth)acrylate, (meth)acrylic acid, N-cyclohexylmaleimide and styrene.

There can be used as well hydroxystyrene homo- or co-polymers or a novolak type phenol resin, for example, poly(hydroxystyrene) and poly(hydroxystyrene-co-vinylcyclohexanol), a novolak resin, a cresol novolak resin, and a halogenated phenol novolak resin. More specifically, it includes, for example, the methacrylic acid copolymers, the acrylic acid copolymers, the itaconic acid copolymers, the crotonic acid copolymers, the maleic anhydride co-polymers, for example, with styrene as a co-monomer, and maleic acid copolymers, and partially esterified maleic acid copolymers each described in, for example, JP 59-4461 5-B4 (the term “JP-B4” as used herein refers to an examined Japanese patent publication), JP 54-34327-B4, JP 58-12577-B4, and JP 54-25957-B4, JP 59-53836-A, JP 59-71 048-A, JP 60-159743-A, JP 60-258539-A, JP 1-152449-A, JP 2-199403-A, and JP 2-199404-A, and which copolymers can be further reacted with an amine, as e.g disclosed in U.S. Pat. No. 5,650,263; further, a cellulose derivative having a carboxyl group on a side chain can be used, and particularly preferred are copolymers of benzyl(meth)acrylate and (meth)acrylic acid and copolymers of benzyl(meth)acrylate, (meth)acrylic acid and other monomers, for example as described in U.S. Pat. No. 4,139,391, JP 59-44615-B4, JP 60-159743-A and JP 60-258539-A.

With respect to those having carboxylic acid groups among the above organic binder polymers, it is possible to react some or all of the carboxylic acid groups with glycidyl(meth)acrγlate or an epoxy(meth)acrylate to obtain photopolymerizable organic binder polymers for the purpose of improving the photosensitivity, coating film strength, the coating solvent and chemical resistance and the adhesion to the substrate. Examples are disclosed in, JP 50-34443-B4 and JP 50-34444-B4, U.S. Pat. No. 5,153,095, by T. Kudo et al. in J. Appl. Phys., Vol. 37 (1998), p. 3594-3603, U.S. Pat. No. 5,677,385, and U.S. Pat. No. 5,650,233.

Among these various kinds of alkali-soluble binders, acrylic acid homo- and copolymers as well as methacrylic acid homo- and copolymers are particularly preferred.

The weight-average molecular weight of the binders is preferably 500 to 1′OOOOOO, e.g. 3′0OO to 10OOOOO₁ more preferably 5′00O to 400O00.

The content of the alkali-soluble binder in the dye-containing curable resin is preferably from 10 to 90% by weight, more preferably from 20 to 80% by weight, and particularly preferably from 30 to 70% by weight, based on the total solid content of the composition.

The dyes of formula (1) are known for the most part and can be synthesized according to well-known methods, for example as described by D. Woehrle et al. in Dyes and Pigments 18(2), 9′-102 (1992).

In formula (1) any radical denoting alkyl may be a straight-chain or branched alkyl radical that may be substituted by one or more hydroxy groups or mercapto groups.

Examples of alkyl groups include methyl, ethyl, n-propyl, isopropyl, n-butyl, isobutyl, sec-butyl, tert-butyl, n-pentyl, isopentyl, neopentyl, n-hexyl, n-heptyl, n-octyl, isooctyl, n-decyl and n-dodecyl.

Substituted alkyl groups include, for example, 2-hydroxyethyl, 2-hydroxypropyl, 4-hydroxybutyl, 2-mercaptoethyl, 2-mercaptopropyl and 4-mercaptbutyl.

The aryl radicals designated Ri to R₄ may have from 5 to 24, especially from 6 to 14, carbon atoms and may be substituted, for example, by hydroxy or mercapto groups

Examples of suitable aryl groups include phenyl, tolyl, mesityl, isityl, 2-hydroxyphenyl, 4-hydroxyphenyl, 2-mercaptphenyl, 4-mercaptophenyl, naphthyl and phenanthryl.

Aralkyl groups as Ri to R₄ may have from 6 to 30, especially from 7 to 12, carbon atoms and may be unsubstituted or substituted by one or more hydroxy or mercapto groups.

Examples of suitable aralkyl groups include benzyl, 2-phenylethyl, tolyl methyl and mesityl methyl.

Preferably, the composition according to the invention contains a dye of formula (1), wherein Ri, R₂, R₃ and R₄ are d-Cˆalkyl or Ci-Ci₂hydroxyalkyl.

Particularly preferred are compounds of formula (1), wherein R₁, R₂, R₃ and R₄ are methyl, ethyl, isopropyl, n-butyl, 2,4-dimethylpentyl-(3) or 2-hydroxyethyl.

The dyes (101) and (102) are the most preferred compounds of formula (1)

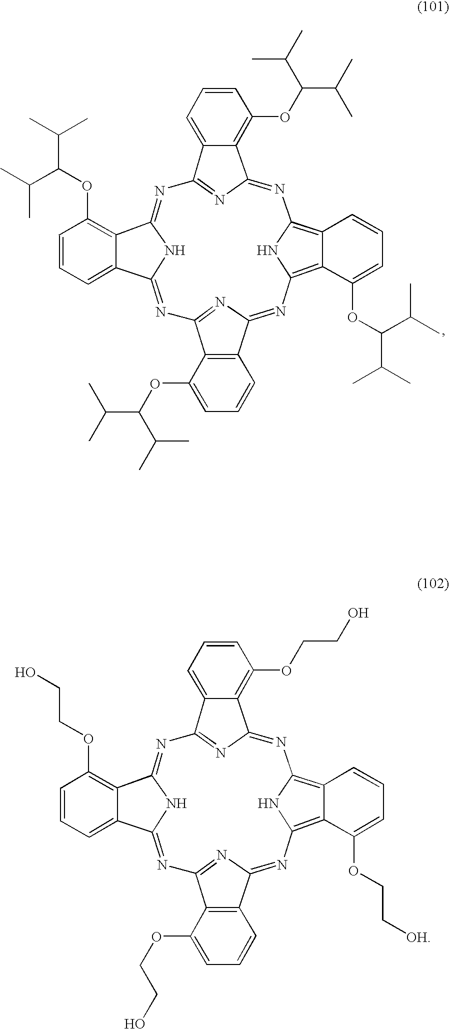

The dye (102) is novel and represents a further embodiment of the invention

The content of the dye of formula (1) in the dye-containing curable resin is preferably from 1 to 50% by weight, more preferably from 3 to 40% by weight, and particularly preferably from 5 to 30% by weight, based on the total solid content of the composition.

In the case where the dye-containing curable composition is constituted as a positive type resist, the composition may contain a photosensitive compound like, for example, a naphthoquinone diazide.

In the case where a negative resist type dye-containing curable composition is constituted, the composition purposively contains a photopolymerisable vinyl compound and a photopolymerisation initiator.

Accordingly, the invention further relates to a composition containing
(A) an alkali-soluble binder,
(B) a phthalocyanine dye of formula (1) as described above.
(C) a photopolymerisable vinyl compound different from component (A) and
(D) a photoinitiator.

Photopolymerisable vinyl compounds are well known to the person skilled in the art.

These monomers contain at least one ethylenic double bond and usually have a boiling point of 100° C. or more.

Examples for suitable photopolymerisable vinyl compounds are polyethylene glycol monoacrylate, polyethylene glycol monomethacrylate, polypropylene glycol monoacrylate, polypropylene glycol monomethacrylate, phenoxyethyl acrylate, phenoxyethyl methacrylate, polyethylene glycol diacrylate, polyethylene glycol dimethacrylate, trimethylolpropane triacrylate, trimethylolpropane triamethcrylate, neopentylglycol diacrylate, neopentylglycol dimethacrylate, pentaerythritol triacrylate, pentaerythritol triamethcrylate, pentaerythritol tetraacrylate, pentaerythritol tetramethcrylate, dipentaerythritol pentaacrylate, dipentaerythritol pentamethacrylate, dipentaerythritol hexaacrylate, dipentaerythritol hexamethacrylate, tri(acryloyloxyethyl)isocyanurate.

Preferred photopolymerisable vinyl compounds are dipentaerythritol pentaacrylate and dipentaerythritol pentamethacrylate.

The total content of the photopolymerisable vinyl compound (C) in the dye-containing curable composition is, while it varies depending on the material thereof, from 5 to 70% by weight, preferably from 5 to 50% by weight, and particularly preferably from 7 to 30% by weight, based on the solid content of the composition.

Suitable photoinitiators (D) are also well known to the person skilled in the art and are preferably selected from halomethyloxadiazols, halomethyl-s-triazines, 3-arylsubstituted coumarins, benzophenones, acetophenones, cyclopentadiene-benzene-iron complexes, oxime esters and oximes.

Suitable photoinitiators (D) are described, for example, in GB 2339571, U.S. Pat. No. 6,485,885, GB 2358017, GB 2357293, WO 02/100903, J. Photopolym. Sci. Technol. 15, 51-57 (2002), IP.com. Journal IPCOM 000012462D, 3(6), 101-109 (2003), US 2004/0102548 and US 2004/0102673.

Preferred photoinitiators (D) are benzophenones of the formula 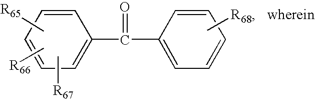
Res, R₆₆ and R₆₇ independently of one another are hydrogen, CrC₄-alkyl, CᵣC₄-halogenalkyl, CrC₄-alkoxy, chlorine or N(d-C₄-alkyl)₂;
R₆₈ is hydrogen, CᵣC₄-alkyl, CᵣC₄-halogenalkyl, phenyl, N(Ci-C₄-alkyl)₂, COOCH₃, 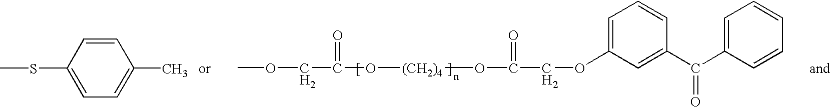
n is 2-10.

Specific examples are ESACURE TZT® available from Lamberti, (a mixture of 2,4,6-trimethylbenzophenone and 4-methylbenzophenone) and DAROCUR® BP (benzophenone).

Further preferred photoinitiators (D) are alpha-hydroxy ketones, alpha-alkoxyketones or alpha-aminoketones of the formula 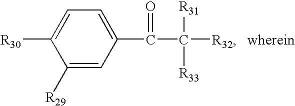
R₂₉ is hydrogen or Ci-Ci₈-alkoxy;
R₃_{⁰} is hydrogen, CrCi₈-alkyl, Ci-Ci₂hydroxyalkyl Ci-Ci₈-alkoxy, —OCH₂CH₂—OR₄₇, morpholino, Ci-Ci₈alkyl-S—, a group H₂C═CH—, H₂C═C(CH₃)—, 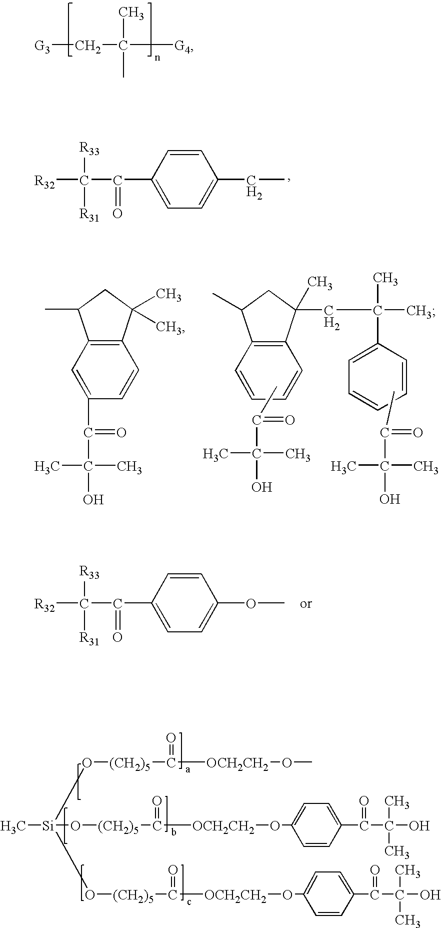
a, b and c are 1-3;
n is 2-10;
G₃ and G₄ independently of one another are end groups of the polymeric structure, preferably hydrogen or methyl;
R₄₇ is hydrogen, 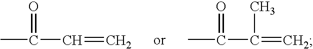
R₃i is hydroxy, CrCi₆-alkoxy, morpholino, dimethylamino or —O(CH₂CH₂O)ₘ—CrCi₆-alkyl;
R₃₂ and R₃₃ independently of one another are hydrogen, CrC₆-alkyl, CᵣCi₆-alkoxy or —O(CH₂CH₂O)ₘ—Ci-Ci₆-alkyl; or unsubstituted phenyl or benzyl; or phenyl or benzyl substituted by CᵣCi₂-alkyl; or R₃₂ and R₃₃ together with the carbon atom to which they are attached form a cyclohexyl ring;
m is 1-20;
with the proviso that R₃i, R₃₂ and R₃₃ not all together are CrCi₆-alkoxy or —O(CH₂CH₂O)ₘ—Ci-Ci₆-alkyl.

Specific examples are 1-hydroxy-cyclohexyl-phenyl-ketone, a mixture of 1-hydroxycyclohexyl-phenyl-ketone with benzophenone, 2-methyl-1[4-(methylthio)phenyl]-2-morpholinopropan-1-one, 2-benzyl-2-dimethylamino-1-(4-morpholinophenyl)-butanone-1,2-dimethylamino-2-(4-methyl-benzyl)-1-(4-morpholin-4-yl-phenyl)-butan-1-one, 1-[4-(2-hydroxyethoxy)-phenyl]-2-hydroxy-2-methyl-1-propan-1-one, 2,2-dimethoxy-1,2-diphenylethan-1-one, 2-hydroxy-2-methyl-1-phenyl-propan-1-one, 2-hydroxy-1-{4-[4-(2-hydroxy-2-methyl-propionyl)-benzyl]-phenyl}-2-methyl-propan-1-one, 2-benzyl-1-(3,4-dimethoxy-phenyl)-2-dimethylamino-butan-1-one, 2-hydroxy-1-{4-[4-(2-hydroxy-2-methyl-propionyl)-phenoxy]-phenyl}-2-methyl-propan-1-one,

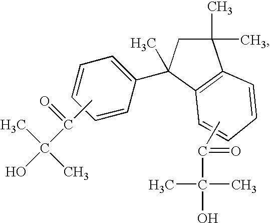

ESACURE KIP provided by Fratelli Lamberti and 2-hydroxy-1-{1-[4-(2-hydroxy-2-methyl-propionyl)-phenyl]-1,3,3-trimethyl-indan-5-yl}-2-methyl-propan-1-one.

Further preferred photoinitiators (D) are acylphosphine oxides of the formula 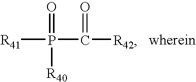
R₄₀ and R₄₁ independently of one another are unsubstituted CrC₂o-alkyl, cyclohexyl, cyclopentyl, phenyl, naphthyl or biphenylyl; or CrC₂o-alkyl, cyclohexyl, cyclopentyl, phenyl, naphthyl or biphenylyl substituted by halogen, CrCi₂-alkyl, CrCi₂-alkoxy, CᵣCi₂alkylthio or NRS₂RsS, or R₄₀ and R41 are independently of one another —(CO)R₄₂;
R₅₂ and R₅₃ independently of one another are hydrogen, unsubstituted CᵣCi₂-alkyl or CᵣCi₂-alkyl substituted by OH or SH wherein the alkyl chain may be interrupted by one to four oxygen atoms; or R₅₂ and R₅₃ independently of one another are C₂-Ci₂-alkenyl, cyclopentyl, cyclohexyl, benzyl or phenyl;
R₄₂ is unsubstituted cyclohexyl, cyclopentyl, phenyl, naphthyl or biphenylyl, or cyclohexyl, cyclopentyl, phenyl, naphthyl or biphenylyl substituted by halogen, CrC₄-alkyl and/or d-C₄-alkoxy; or R₄₂ is a 5- or 6-membered heterocyclic ring having an S atom or N atom;

Specific examples are bis(2,4,6-trimethylbenzoyl)-phenylphosphine oxide, 2,4,6-trimethylbenzoyl-diphenyl-phosphine oxide, bis(2,6-dimethoxybenzoyl)-2,4,4-trimethylpentylphosphine oxide,

Further preferred photoinitiators (D) are titanocenes of the formula 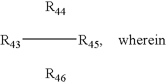
R₄₃ and R₄₄ independently of one another are cyclopentadienyl optionally mono-, di-, or tri-substituted by d-ds-alkyl, CᵣCi₈-alkoxy, cyclopentyl, cyclohexyl or halogen;
R₄₅ and R₄₆ are phenyl having at least one F or CF₃ substituent in ortho position to the Ti—C bond and having at least a further substituent which is unsubstituted pyrrolinyl or polyoxaalkyl or which is pyrrolinyl or polyoxaalkyl substituted by one or two Ci-Ci₂-alkyl, di(Ci-Ci₂-alkyl)aminomethyl, morpholinomethyl, C₂-C₄-alkenyl, methoxymethyl, ethoxymethyl, trimethylsilyl, formyl, methoxy or phenyl; or
R₄₅ and R₄₆ are 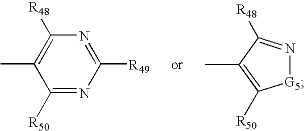
G₅ is O, S, or NR₅₁;
R₄₈, R₄₉ and R₅₀ independently of one another are hydrogen, halogen, C₂-Ci₂-alkenyl, CrCi₂alkoxy, C₂-Ci₂-alkoxy interrupted by one to four oxygen atoms, cyclohexyloxy, cyclopentyloxy, phenoxy, benzyloxy, unsubstituted phenyl or biphenyl or phenyl or biphenyl substituted by CrC₄-alkoxy, halogen, phenylthio or CrC₄-alkylthio, with the proviso that R₄₈ and R₅₀ are not both hydrogen and that with respect to the residue 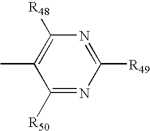
at least one substituent R₄₈ or R₅₀ is Ci-Ci₂alkoxy or Ci-Ci₂alkoxy interrupted by one to four oxygen atoms, cyclohexyloxy, cyclopentyloxy, phenoxy or benzyloxy; and
R₅i is CrC₈alkyl, phenyl or cyclophenyl.

Specific examples are bis(.eta.5-2,4-cyclopentadien-1-yl)-bis(2,6-difluoro-3-(1H-pyrrol-1-yl)-phenyl)-titanium and bis(2,6-difluorophenyl)bis[(1,2,3,4,5-eta)-!-methyl-2,4-cyclopentadien-1-yl]-titanium.

Further preferred photoinitiators (D) are phenylglyoxalates of the formula 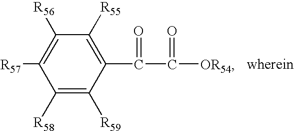
R₅₄ is hydrogen, Ci-Ci₂-alkyl or 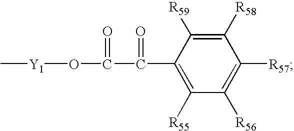
R55, Rse, R57, R58 and R₅₉ independently of one another are hydrogen, unsubstituted Ci-d₂-alkyl or Ci-Ci₂-alkyl substituted by OH, CrC₄-alkoxy, phenyl, naphthyl, halogen or CN;
wherein the alkyl chain optionally is interrupted by one or more oxygen atoms; or R₅₅, R₅₆, R₅₇, R₅₈ and R₅₉ independently of one another are CrC₄-alkoxy, Ci-C₄-alkythio or NR₅₂R₅3;
R₅₂ and R₅₃ independently of one another are hydrogen, unsubstituted CᵣCi₂-alkyl or Ci-Ci₂-alkyl substituted by OH or SH wherein the alkyl chain optionally is interrupted by one to four oxygen atoms; or R₅₂ and R₅₃ independently of one another are C₂-Ci₂-alkenyl, cyclopentyl, cyclohexyl, benzyl or phenyl; and
Yi is Ci-Ci₂-alkylene optionally interrupted by one or more oxygen atoms.

A specific example is oxo-phenyl-acetic acid 2-[2-(2-oxo-2-phenyl-acetoxy)-ethoxy]-ethyl ester.

Further preferred photoinitiators (D) are oxime esters of the formula 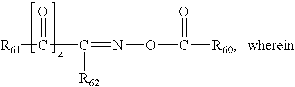
z is O or 1;
R₆_{⁰} is hydrogen, C₃-C₈cycloalkyl; d-Cˆalkyl which is unsubstituted or substituted by one or more halogen, phenyl and/or CN; or R₆₀ is C₂-C₅alkenyl; phenyl which is unsubstituted or substituted by one or more CrC₆alkyl, halogen, CN, OR₆₃, SR₆_{⁴} and/or NR₆₅R₆₆; or R₆₀ is d-C₈alkoxy, benzyloxy; or phenoxy which is unsubstituted or substituted by one or more CᵣC₆alkyl and/or halogen;
R₆i is phenyl, naphthyl, benzoyl or naphthoyl, each of which is substituted 1 to 7 times by halogen, CᵣCi₂alkyl, C₃-C₈cycloalkyl, benzyl, phenoxycarbonyl, C₂-Ci₂alkoxycarbonyl, OR₆₃, SR₆₄SOR₆₄, SO₂R₆₄ and/or NR₆₅R₆₆, wherein the substituents OR₆₃, SR₄ and NR₆₅R₆₆ optionally form 5- or 6-membered rings via the radicals R₆₃, R₆₄, R₆₅ and/or R₆₆ with further substituents on the phenyl or naphthyl ring; or each of which is substituted by phenyl or by phenyl which is substituted by one or more OR₆₃, SR₆₄ and/or NR₆₅R₆₆;
or R₆₁ is thioxanthylor 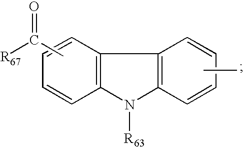
R₆₂ is hydrogen; unsubstituted CᵣC_{2O}alkyl or CᵣC_{2O}alkyl substituted by one or more halogen, OR₆₃, phenyl; or is C₃-C₈cycloalkyl; phenyl which is unsubstituted or substituted by one or more CᵣC₆alkyl, phenyl, halogen, OR₆₃, SR₆₄ and/or NR₆₅R₆₆; or is C₂-C₂₀alkanoyl or benzoyl which is unsubstituted or substituted by one or more CrC₆alkyl, phenyl, OR₆₃, SR₆₄ and/or NR₆₅R₆₆; or is C₂-Ci₂alkoxycarbonyl, phenoxycarbonyl, CN, —CONR₆₅R₆₆, NO₂, CᵣC₄haloalkyl, S(O)yCᵣC₆alkyl; S(O)yphenyl,
y is 1 or 2;
R₆₃ and R₆₄ independently of one another are hydrogen, CrC_{2O}alkyl, C₂-Ci₂alkenyl, C₃-C₈cycloalkyl, phenyl-CᵣC₃alkyl; or are CᵣC₈alkyl which is substituted by —OH, —SH, —CN, CᵣC₈alkanoyl, benzoyl, which is unsubstituted or substituted by one or more CᵣC₆alkyl, halogen, —OH, Ci-C₄alkoxy or Ci-C₄alkylsulfanyl; or are phenyl or naphthyl, each of which is unsubstituted or substituted by halogen, CᵣCi₂alkyl, CᵣCi₂alkoxy, phenyl-CᵣC₃alkyloxy, phenoxy, CrCi₂alkylsulfanyl, phenylsulfanyl, —N(Ci-Ci₂alkyl)₂, diphenylamino;
R₆₅ and R₆₆ independently of one another are independently of each other are hydrogen, CᵣC_{2O}alkyl, C₂-C₄hydroxyalkyl, C₂-Ci_{O}alkoxyalkyl, C₂-C₅alkenyl, C₃-C₈cycloalkyl, phenyl-Ci-C₃alkyl, CᵣC₈alkanoyl, C₃-Ci₂alkenoyl, benzoyl; or are phenyl or naphthyl, each of which is unsubstituted or substituted by CrCi₂alkyl, benzoyl or Ci-Ci₂alkoxy; or R₆₅ and R₆₆ together are C₂-C₆alkylene optionally interrupted by —O— or —NR₆₃— and/or optionally substituted by hydroxyl, CrC₄alkoxy, C₂-C₄alkanoyloxy or benzoyloxy;
R₆7 is CrCi₂alkyl, phenyl or d-Cˆalkylphenyl Specific examples are 1,2-octanedione 1-[4-(phenylthio)phenyl]-2-(O-benzoyloxime), ethanone 1-[9-ethyl-6-(2-methylbenzoyl)-9H-carbazol-3-yl]-1-(O-acetyloxime) and 9H-thioxanthene-2-carboxaldehyde 9-oxo-2-(O-acetyloxime).

A further example of a photoinitiator is Esacure 1001 available from Lamberti:
1-[4-(4-benzoylphenysulfanyl)phenyl]-2-methyl-2-(4-methylphenylsulfonyl)propan-1-one

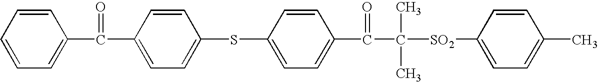

The most preferred photoinitiators are the following compounds:

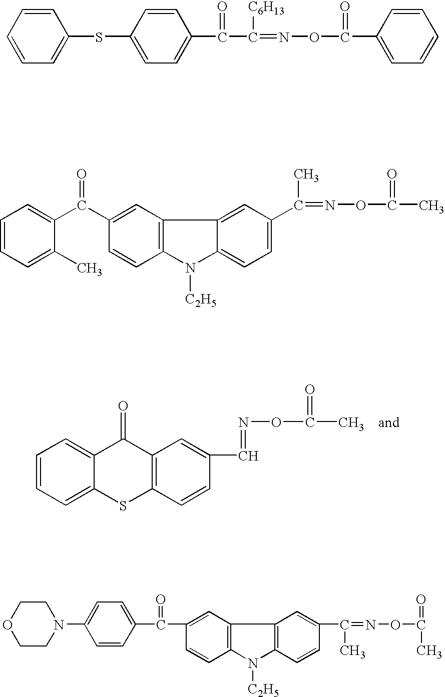

The photoinitiator may be used in combination with a sensitizer and a photostabiliser.

The total content of the photoinitiator is preferably from 0.01 to 10% by weight, preferably from 0.05 to 8% by weight, and particularly preferably from 1 to 5% by weight, based on the solid content of the composition.

Upon preparation of the dye-containing curable composition, a solvent is generally used. The solvent is not particularly limited as far as t satisfies solubility to the respective components and coating property of the dye-containing curable composition and it is preferably selected under particular consideration of the solubility of the alkali-soluble binder, the coating property and the safety.

Suitable solvents include esters, e.g. ethyl acetate, butyl acetate, butyl butyrate and methyl methoxyacetate, ethers like diethylene glycol dimethyl ether, methylcellosolve acetate, butylcarbitol acetate and tetrahydrofurane, ketones, e.g. 2-butanone, cyclopentanone and cyclohexanone, and aromatic hydrocarbons such as toluene and xylene.

Other conventional dyes can be used in combination with the dyes of formula (1), for example azo dyes like the compound of formula (201)

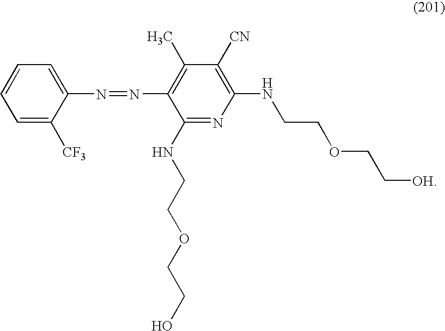

The dyes of formula (1) can also be employed in combination with conventional pigments such as C.I. Pigment Green 36, C.I. Pigment Green 7, C.I. Pigment Red 254, C.I. Pigment Red 177, C.I. Pigment Blue 15:6, C.I. Pigment Yellow 138, C.I. Pigment Yellow 139, C.I. Pigment Yellow 150, and C.I. Pigment Violet 23.

A further object of the invention is a composition containing
(A) an alkali-soluble binder,
(B) a dye of formula (1) as described above, and
(C) a pigment.

Various kinds of additives may be added to the dye-containing curable compositions according to the invention, such as fillers, polymers, surfactants, dispersing agents, adhesion accelerating agents, antioxidants, UV absorbing agents and aggregation preventing agents.

The invention further relates to a process for producing a colour filter comprising the steps of
(a) coating a support layer with the dye-containing composition as described above,
(b) irradiating the coated layer through a mask and
(c) developing the exposed composition to form a pattern.

In the process for producing a colour filter according to the invention, the dye-containing curable composition is coated on a support by conventional coating methods like spin coating, flow coating and roll coating to form a radiation-sensitive composition layer which is then exposed through a prescribed mask pattern, followed by development to form a coloured pattern. Thereafter, thus formed coloured pattern is cured by heating.

As radiation used herein, an ultraviolet ray such as g-line, h-line and i-line is particularly preferred.

Examples of the support include soda glass, Pyrex® glass and quartz glass which are used in a liquid crystal display device or the like, those having a transparent electroconductive film adhered, and a photoelectric conversion element substrate, such as a silicon substrate, and a complementary metallic oxide semiconductor (CMOS), which are used in a solid state image sensing device or the like.

An undercoating layer may be provided, depending on necessity, on the support for improvement of adhesion to the upper layer, prevention of diffusion of substances, and planarisation of the surface of the substrate.

When the dye of formula (1) is used in combination with a pigment, dye and pigment can be applied in different layers in either sequence on the same pixel or they can be applied in different pixels.

The dyes of formula (1) are characterised by excellent thermostability and light stability as well as by outstanding immobilisation of the dye into the final coated layer.

The following Examples serve to illustrate the invention. In the Examples, unless otherwise indicated, parts are parts by weight and percentages are percent by weight. The temperatures are given in degrees Celsius. The relationship between parts by weight and parts by volume is the same as that between grams and cubic centimeters.

General Procedure:

A liquid formulation containing an acrylic acid/acrylate polymeric resin binder, an organic solvent, a photoinitiator, a polymerisable monomer, a dye and optionally a dispersant is homogenized by stirring and filtered over a 0.45 microns Teflon filter. Spin coating of this formulation is performed on glass plates at various spinning speeds in order to achieve various layer thicknesses. Soft bake at 100° C. for 2 min affords the required thin transparent layer.

UV exposure through a mask for 30 sec followed by basic aqueous development and final post bake for 5 min at 240° C. results in a structured pattern.

Formulation A:

6.3 parts Disperbyk® 161 (cationic polyurethane, dispersing agent)
13.8 parts acrylic acid/acrylate resin binder
41.3 parts cyclopentanone
7.5 parts SR 399 (dipentaerytritol pentaacrylate)
0.5 parts 2,4-bis(trichloromethyl)-6-(4-methoxyphenyl)-1,3,5-triazine
   Formulation B:
2.5 parts dye of the formula (201)
26.0 parts cyclopentanone
   Formulation C
0.3 parts dye of the formula (101)
3.0 party Formulation A

### EXAMPLE 1

0.5 g of the dye of the formula (101) are dissolved in 4.4 g of formulation A and applied according to the procedure described above.

At spinning speed of 1000 rpm, layer thickness is 2.88 microns, colour point values are x=0.3037, y=0.4883, Y=64.09.

### EXAMPLE 2

Mixture of two dyes into same formulation:

0.08 g of formulation B is added to 0.14 g of formulation C and applied according to the procedure described above.

At spinning speed of 1000 rpm, colour point values are x=0.3669, y=0.5408, Y=74.75.

### EXAMPLE 3

Two superimposed coatings at the same pixel:

0.3 g of dye of the formula (201) are dissolved in 3.0 g of formulation A and applied according to the procedure described above. The thus coated glass plate is kept for the next spin-coating to be performed on top of the first coating.

0.3 g of dye of the formula (101) are dissolved in 3.0 g of formulation A and applied according to the procedure described above on the glass plate prepared above.

At spinning speeds of 1000 rpm, colour point values are x=0.3478, y=0.6237, Y=55.55.

1. A composition containing
(A) an alkali-soluble binder and
(B) a phthalocyanine dye of formula 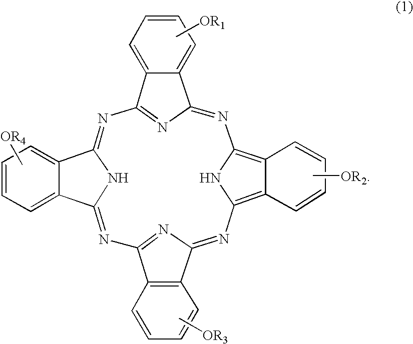
wherein
Ri, R₂, R₃ and R₄ are each independently of the other CrCi₂alkyl which may be unsubstituted or substituted by one or more hydroxy or mercapto groups,
—X—[—O—Y]ₙ-ZH, wherein X and Y are each independently of the other C₂-C₆alkylene, Z denotes oxygen or sulphur and n is a number from 1 to 10,
C₅-C₂₄aryl which may be unsubstituted or substituted by one or more hydroxy or mercapto groups, or
C₆-C₃₀aralkyl which may be unsubstituted or substituted by one or more hydroxy or mercapto groups.

2. A composition according to claim 1 wherein the alkali-soluble binder is an acrylic acid homo- or copolymer or a methacrylic acid homo- or copolymer.

3. A composition according to claim 1 containing a phthalocyanine dye of formula (1) wherein R₁, R₂, R₃ and R₄ are Ci-d₂alkyl or CᵣCi₂hydroxyalkyl.

4. A composition according to claim 1 containing a phthalocyanine dye of formula (1) wherein R₁, R₂, R₃ and R₄ are methyl, ethyl, isopropyl, n-butyl, 2,4-dimethylpentyl-(3) or 2-hydroxyethyl.

5. A composition according to claim 1 additionally containing
(C) a photopolymerisable vinyl compound different from component (A) and
(D) a photoinitiator.

6. A composition according to claim 1 additionally containing
(E) a pigment.

7. A process for producing a colour filter comprising the steps of
(a) coating a support layer with the dye containing composition according to claim 1,
(b) irradiating the coated layer through a mask and
(c) developing the exposed composition to form a pattern.

8. The compound of formula (102) [chemical structure]
